(19) United States
(12) Patent Application Publication
(10) Pub. No.: US 2011/0245123 A1
(43) Pub. Date: Oct. 6, 2011

(54) METHODS OF EMULSIFYING ORGANOSILOXANE POLYMERS

(76) Inventors: Jeremy Wayne Cox, Crescent, KY (US); Rajan Keshav Panandiker, West Chester, OH (US); Julie Ann Menkhaus, Cleves, OH (US); Kerry Andrew Vetter, Cincinnati, OH (US)

(21) Appl. No.: 13/078,047

(22) Filed: Apr. 1, 2011

Related U.S. Application Data
(60) Provisional application No. 61/319,939, filed on Apr. 1, 2010.

(51) Int. Cl. A61K 8/89; A61K 8/06; A61K 8/898; A61Q 5/12; A61Q 5/02; C11D 3/60; A61Q 13/00

(52) U.S. Cl. 510/122; 424/70.12; 424/401; 424/70.122; 514/772; 514/788; 510/515; 512/4

(57) ABSTRACT

Emulsified organosiloxane polymers are useful in consumer product compositions. In one aspect, a method of making a consumer product having an organosiloxane polymer emulsion comprising the steps: providing a surfactant/solvent mixture; providing an organosiloxane polymer; mixing the surfactant/solvent mixture and the organosiloxane polymer to obtain a homogenous silicone premix; and adding to the homogenous silicone premix to a consumer product precursor composition to obtain the consumer product is disclosed.

### CROSS-REFERENCES TO RELATED APPLICATIONS

This application claims priority under 35 U.S.C. §119(e) to U.S. Provisional Application Ser. No. 61/319,939, filed Apr. 1, 2010.

### FIELD OF THE INVENTION

The field of the invention is directed to methods of making emulsified organoxiloxane polymers and consumer products containing the same.

### BACKGROUND OF THE INVENTION

Organosiloxane polymers, and methods of emulsifying the same, have been reported. U.S. Pat. Nos. 6,815,069; 7,153,924; 7,321,019; and 7,427,648. However, many emulsifying methods are conducted at high shear. High shear often requires a high energy or capital investment. High shear may also degrade other actives when making consumer product compositions having organosiloxane polymer emulsions. Many methods to emulsify silicones are conducted under high energy density mixing conditions (e.g., 10⁵ to 10¹⁰ W/kg). These methods may be limited in their ability to deliver relatively small emulsions droplets. There is a need for methods of emulsifying organosiloxane polymers at lower shear and ambient temperatures. There is need to have nano-sized organosiloxane polymer emulsion droplets in consumer product compositions and without degrading other actives in the composition. Lastly, there is a need to emulsify organosiloxanes with cationic charged emulsifiers—particularly in the context of making liquid fabric softeners that have a low pH and cationic softening actives.

### SUMMARY OF THE INVENTION

The present invention attempts to address one more of these needs but providing, in a first aspect of the invention, a method of making a consumer product having an organosiloxane polymer emulsion comprising the steps: providing a surfactant/solvent mixture; providing an organosiloxane polymer; mixing the surfactant/solvent mixture and the organosiloxane polymer to obtain a homogenous silicone premix; and adding to the homogenous silicone premix to a consumer product precursor composition to obtain the consumer product.

Another aspect of the invention provides for a method of making a consumer product having an organosiloxane polymer emulsion comprising the steps of: providing a surfactant/solvent mixture; providing an organosiloxane polymer; mixing the surfactant/solvent mixture and the organosiloxane polymer to obtain a homogenous silicone premix; mixing the homogenous silicone premix with water to obtain an organosiloxane polymer emulsion; and adding the organosiloxane polymer emulsion to a consumer product precursor composition to obtain the consumer product.

Another aspect of the invention provides for a method of making a consumer product having an organosiloxane polymer emulsion comprising the steps: providing a surfactant/solvent mixture; providing an organosiloxane polymer; mixing the surfactant/solvent mixture and the organosiloxane polymer to obtain a homogenous silicone premix; and adding the homogenous silicone premix to a consumer product precursor composition to obtain the consumer product.

Another aspect of the invention provides for a method of making a consumer product having an organosiloxane polymer emulsion comprising the steps: providing a homogenous silicone premix comprising a organosiloxane polymer, solvent, and surfactant; adding to the homogenous silicone premix to a consumer product precursor composition under ambient temperature conditions; and mixing the premix and the precursor under low energy density conditions to form the consumer product, wherein the consumer product comprises oil-in-water organosiloxane polymer emulsion droplets such that the mean droplet diameter (as measured by volume distribution base) is below 1 micron.

Another aspect of the invention provides for a method of making a consumer product comprising the step: mixing a polyorganosiloxane with a high hydrogen bonding solvent (i.e., having a high Hansen Solubility Parameter, i.e., greater than 10.0 for Hydrogen bonding capacity, δ_{H}), preferably the solvent is ethanol, to obtain a binary homogenous silicone premix; slowly adding the binary homogenous silicone premix to an aqueous surfactant mixture to obtain a polyorganosiloxane emulsion, preferably wherein the aqueous surfactant mixture comprises a surfactant/polyorganosiloxane weight ratio from 1:1 to about 1:10, respectively; and optionally mixing the aqueous surfactant mixture while the homogenous silicone premix is added to the aqueous surfactant mixture.

Another aspect of the invention provides for a method of making a consumer product comprising the steps: mixing a polyorganosiloxane with a high hydrogen bonding solvent (i.e., having a high Hansen Solubility Parameter), preferably the solvent is ethanol, to obtain a binary homogenous silicone premix; slowly adding a surfactant to the binary homogenous silicone premix to obtain a homogenous silicone premix, preferably wherein the aqueous surfactant mixture comprises a surfactant/polyorganosiloxane weight ratio of from about 1:1 to about 1:30, alternatively from 1:1 to 1:10; adding water to the homogenous silicone premix to obtain a polyorganosiloxane emulsion; and optionally mixing the homogenous silicone premix while the water is added to obtain the emulsion.

### DETAILED DESCRIPTION OF THE INVENTION

### Homogenous Silicone Premix to Make Emulsions

One aspect of the invention provides for a method of making a homogenous silicone premix. Generally the method comprises the step of mixing surfactant(s) and solvent(s) to form a surfactant/solvent mixture. Thereafter, the surfactant/solvent mixture is added to the organosiloxane polymer to form the homogenous silicone premix.

In one iteration of the invention an organosiloxane emulsion is formed by adding water to the homogenous silicone premix. This emulsion may then be added to a consumer product precursor composition to make a final consumer product composition having an organosiloxane emulsion. A consumer product precursor composition comprises water and at least one consumer product active. The active and amount of water will depend upon the consumer product.

In another iteration, the homogenous silicone premix is added directly to the consumer product precursor composition to make the consumer product composition. The emulsion is made using the water that exists in the consumer product precursor composition. In other words, a separate step of making an emulsion and then having the emulsion added to the consumer product precursor composition is avoided. Such an approach lends itself to so called late product differentiation manufacturing methods.

One or more of these steps may be conducted under ambient temperature conditions and/or with low energy density mixing conditions. “Ambient temperature” means from 7° C. to 32° C., alternatively from 15° C. to 25° C., alternatively from 19° C. to 22° C., alternatively combinations thereof. The temperature is taken of the liquid compositions described. “Low energy density mixing conditions” means lower than 10⁵ W/kg, alternatively less than 10⁴ W/kg, alternatively less than 10³ W/kg, alternatively less than 10² W/kg, alternatively less than 10¹ W/kg, alternatively from 10¹ W/kg to 10⁵ W/kg, alternatively combinations thereof. Examples of such a mixing method may include static mixing, low pressure drop orifices, impeller agitation, and single stage mills.

### Surfactant

One aspect of the invention provides for providing one or more surfactants and mixing the surfactant(s) with a solvent to provide a surfactant/solvent mixture, alternatively an aqueous surfactant mixture. Although the exact ratio of surfactant to solvent may vary depending upon the surfactant and solvent used, in one embodiment, the ratio, on a weight basis, of surfactant to solvent is from about 4:1 to about 1:1, alternatively from 5:1 to about 1:2, alternatively from 3:1 to about 1:1, alternatively 2:1 to about 1:3, respectively, alternatively combinations thereof.

Surfactants suitable for the methods described herein are known per se, such as anionic, nonionic, cationic and ampholytic surfactants suitable for emulsifying. Non-limiting examples of surfactants suitable for the methods herein are described may include as anionic, nonionic, cationic and ampholytic surfactants. Non-limiting examples of nonionic surfactants include: Polyvinyl alcohols which still have from 5 to 50%, preferably from 8 to 20%, of vinyl acetate units, having a degree of polymerization from 500 to 3000; Alkylpolyglycol ethers, preferably those having 8 to 40 EO units and alkyl radicals of 8 to 20 carbon atoms; Alkylarylpolyglycol ethers, preferably those having 8 to 40 EO units and 8 to 20 carbon atoms in the alkyl and aryl radicals; Linear organo(poly)siloxanes containing polar groups, in particular those having alkoxy groups having up to 24 carbon atoms and/or up to 40 EO and/or PO groups. Examples of cationic surfactants include: Quaternary alkyl- and alkylbenzeneammonium salts, in particular those whose alkyl groups have 6 to 24 carbon atoms, in particular the halides, sulfates, phosphates and acetates; Alkylpyridinium, alkylimidazolinium and alkyloxazolinium salts, in particular those whose alkyl chain has up to 18 carbon atoms, especially the halides, sulfates, phosphates and acetates. Polyvinyl alcohol and alkylpolyglycol ethers may also be examples.

In one embodiment, the surfactant is a nonionic secondary alcohol ethoxylate. One example of such a surfactant is one sold under the product name of TERGITOL™ (TERGITOL™ 15S-12 and TERGITOL™ 15S-5) from DOW CHEMICAL (and mixtures thereof).

In one embodiment, the surfactant is a quaternary ammonium compound. Preferably, the quaternary ammonium compound is a hydrocarbyl quaternary ammonium compound of formula (II):

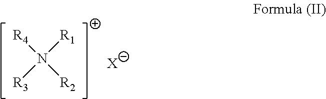

wherein R₁ comprises a C₁₂ to C₂₂ hydrocarbyl chain, wherein R₂ comprises a C₆ to C₁₂ hydrocarbyl chain, wherein R₁ has at least two more carbon atoms in the hydrocarbyl chain than R₂, wherein R₃ and R₄ are individually selected from the group consisting of C₁-C₄ hydrocarbyl, C₁-C₄ hydroxy hydrocarbyl, benzyl, —(C₂H₄O)ₓH where x has a value from about 1 to about 10, and mixtures thereof, and X⁻ is a suitable charge balancing counter ion, in one aspect X⁻ is selected from the group consisting of Cl⁻, Br⁻, I⁻, methyl sulfate, toluene, sulfonate, carboxylate and phosphate
or a polyalkoxy quaternary ammonium compound of Formula (III)

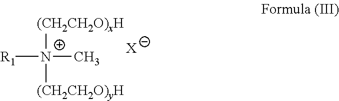

wherein x and y are each independently selected from 1 to 20, and wherein R1 is C6 to C22 alkyl, preferably wherein the aqueous surfactant mixture comprises a surfactant/polyorganosiloxane weight ratio of from about 1:1 to about 1:10 and X⁻ is a suitable charge balancing counter ion, in one aspect X⁻ is selected from the group consisting of Cl⁻, Br⁻, I⁻, methyl sulfate, toluene, sulfonate, carboxylate and phosphate.

Examples of such quaternary ammonium compounds are hydrogenated tallow alkyl (2-ethylhexyl), dimethyl ammonium methyl sulfates sold under the product name, for example, ARQUAD® HTL8-MS and cocoalkyl bis(polyhydroxyethyl)methyl ammonium chloride, sold under the product name, for example, ETHOQUAD® C 25 and ETHOQUAD® C12 all available from AKZO NOBEL of Chicago, Ill.

In another embodiment, the surfactant is a quaternary ammonium containing compounds. Exemplary quaternary ammonium compounds include alkylated quaternary ammonium compounds, ring or cyclic quaternary ammonium compounds, aromatic quaternary ammonium compounds, diquaternary ammonium compounds, alkoxylated quaternary ammonium compounds, amidoamine quaternary ammonium compounds, ester quaternary ammonium compounds, and mixtures thereof. Examples are described in US 2004/0204337.

In one embodiment, a silicone based surfactant is used.

Using ethanol as the solvent, the following surfactants (and levels thereof) were assessed.

| SLM 21-214 | Ethanol | | Surfactant | Mean Particle | | |
|---|---|---|---|---|---|---|
| Level (wt %) | Level (wt %) | Surfactant | Level (wt %) | Size | | Stability |
| 20 | 2.5 | Lauryl trimethyl- | 1.75 | not tested | | Did not emulsify |
| | | ammonium chloride | | | | |
| 20 | 2.5 | Ethoquad C/12-75 | 1.75 | 16.4 | μm | Stable |
| 20 | 2.5 | Ethoquad C/25 | 1.75 | not tested | | Did not emulsify |
| 20 | 2.5 | Ethoquad 18 25 | 1.75 | not tested | | Did not emulsify |
| 20 | 2.5 | Ethoquad O/12 | 1.75 | 9.1 | μm | Stable |
| 10 | 1.25 | Arquad HTL8-MS | 3.5 | 0.288 | μm | Stable |
| 10 | 0 | Arquad HTL8-MS | 3.5 | 58.3 | μm | Stable |
| 10 | 1.25 | Arquad HTL8-MS | 1.75 | 39.7 | μm | Stable |
| 20 | 2.5 | Arquad HTL8-MS | 1.75 | 0.341 | μm | Stable |

The Particle Size analysis was done on a Horiba LA-930 under standard conditions using a Relative Refractive Index (RRI) of 1.05.

### Solvent

One aspect of the invention provides for providing one or more surfactants and mixing the surfactant(s) with a solvent to provide a surfactant/solvent mixture. The solvent may include water, C₁-C₆ alcohols, ethanol, propanol, isopropanol, n-propanol, n-butanol, t-butanol, glycerol, ethylene glycol, trimethylene glycol, tetramethylene glycol, pentamethylene glycol, hexamethylene glycol, diethylene glycol, triethylene glycol, propylene glycol, dipropylene glycol and C₁-C₄ alkyl monoethers of ethylene glycol, propylene glycol, and dipropylene glycol, sorbitol, alkane diols such as 1,2 propanediol, 1,3 propanediol, 2,3-butanediol, 1,4-butanediol, 1,3-butanediol, 1,5-pentanediol, and 1,6 hexanediol; phenylethyl alcohol, 2-methyl 1,3-propanediol, hexylene glycol, sorbitol, polyethylene glycols, 1,2-hexanediol, 1,2-pentanediol, 1,2-butanediol, 1,4-cyclohexanedimethanol, pinacol, 2,4-dimethyl-2,4-pentanediol, 2,2,4-trimethyl-1,3-pentanediol (and ethoxylates), 2-ethyl-1,3-hexanediol, 2-hexyl-1-decanol, 2-butyl-1-octanol, phenoxyethanol (and ethoxylates), glycol ethers such as butyl carbitol, monobutyl ether, dipropylene glycol n-butyl ether, tripropylene glycol n-Butyl ether, Propylene glycol Diacetate, Propylene glycol methyl ether acetate, tripropylene glycol n-Butyl ether, or combinations thereof. Additional solvents are described in U.S. Pat. No. 5,895,504 incorporated by reference.

In one embodiment, the solvent is a high hydrogen bonding solvent (i.e., having a Hansen Solubility Parameter of greater than 10.0 for Hydrogen bonding capacity, δ_{H}), HansenIn one embodiment, the solvents are selected from C1-C6 alcohols, preferably ethanol or isopropanol.

In one embodiment, the solvent may comprise a fatty acid or a fatty acid triglyceride or their derivatives, or a mixture thereof (e.g., fatty acid triglyceride having an average chain length of the fatty acid moieties of from 10 to 14 carbon atoms). In one embodiment the solvent may include a fat derivative such as isopropyl palmitate, isopropyl myristate, branched fatty alcohols from about 6 to about 18 carbon atoms in their alkyl chain. In one embodiment, the branched fatty alcohols are selected from butyl 2 ethyl hexanol, 2 butyl octanol, and 2 hexanol. Examples of such materials are commercially available from Sasol Chemicals, Johannesburg, South Africa under the trade name Isofol® 12, Isofol® 16 or Isalchem® 145 (c14-15 mono-branched). In another embodiment, the solvents may comprise paraffin and isoparaffin of boiling point between 150° C. and 550° C.

Water or ethanol are preferred solvents. A solvent consisting essentially of water is most preferred given the low cost and low flammability.

Once the surfactant/solvent mixture is formed, the surfactant/solvent mixture is mixed to the organosiloxane polymer to form a homogenous silicone premix. Preferably the premix should stand, even if for a few seconds. Standing time may include at least 3 seconds, or at least 5 seconds, or at least 10 seconds, or at least 15 seconds, or at least 20 seconds, or at least one minute, or from 5 seconds to 48 or more hours, or from 5 seconds to 24 hours, or from 10 seconds to 8 hours, or from 20 seconds to 1 hour, or combinations thereof, to allow diffusion to create the homogenous silicone premix. One skilled in the art can determine when a homogenous solution is formed because the homogenous silicone premix will become transparent or substantially transparent.

Without wishing to be bound by theory, the homogenous silicone premix provides improved emulsification by providing a relatively smaller emulsion droplet diameter—even under ambient and/or low energy density mixing conditions.

The use of a spectrophotometer may be one non-limiting method to determine whether the silicone premix is homogenous. The silicone premix may be considered homogenous when the percent transmittance of light is greater than about 50% using a 1 centimeter cuvette at a wavelength of 570 nanometers. Alternatively, the transparency of the composition may be measured as having an absorbance (A) at 570 nanometers of less than about 0.3 which is in turn equivalent to percent transmittance of greater than about 50% using the same cuvette as above. The relationship between absorbance and percent transmittance is: Percent Transmittance=100 (1/inverse log A). Of course the exact percent/absorbance will depend upon factor such as the concentration of organosiloxane polymer, and type/concentration of solvent and surfactant.

### Binary Homogenous Silicone Premixes

In one aspect of the invention the organosiloxane polymers are combined with a solvent, such as ethanol, to make a binary homogenous silicone premix. The weight ratio of silicone to solvent is 50:50 to 98:2, alternatively from 50:50 to 95:5, respectively.

In one embodiment, the method comprises adding the binary homogenous silicone premix to an aqueous surfactant mixture to obtain an organosiloxane emulsion.

In another embodiment, the method comprises adding a surfactant to the binary homogenous silicone premix to obtain a homogenous silicone premix. An emulsion may be easily made by adding water to the homogenous silicone premix. Without wishing to be bound by theory, the water induces a phase inversion to produce an emulsion.

### Organosiloxanes Polymer Premixes

Those organosiloxanes polymers that are hydrophilic are preferably used because they are easier to emulsify, possibly even in the absence of surfactant. Examples of such organosiloxanes polymers are detailed below. However, a high degree of hydrophilicity can make the organosiloxane polymers very hygroscopic, such that when the organosiloxane polymer is added to water at ambient temperature, and without wishing to be bound by theory, two mechanisms are likely simultaneously occurring: emulsification and absorption of water. This absorption of water may dramatically alter the organosiloxane rheology rapidly, possibly preventing emulsification altogether by promoting elastic elongation behaviors. This so-called gellation behavior happens quickly, in a time frame that is shorter than the total time of an emulsification reaction.

Without wishing to be bound by theory, when surfactant is homogeneously dispersed throughout the surfactant premix, the surfactant molecules have locality next to the organosiloxane molecules, likely enabling them to modulate or buffer their absorption of water and prevent the rapid rheology change. In a contrasting heterogenous mixture, the surfactant is concentrated in a separate phase away from the majority of the organosiloxane polymer molecules, so it can provide no such benefit.

The solvent should be chosen to have solubility characteristics which bridge the differences between the surfactant and organosiloxane. A most preferred solvent is one where the surfactant has a higher intermolecular attraction with the solvent than the solvent has with the organosiloxane.

Upon adding the surfactant/solvent mixture to the organosiloxane polymer, the resulting homogenous silicone premix comprises from about 75% to about 97% of organosiloxane polymer by weight of the homogenous silicone premix. One skilled in the art will readily adjust the amounts of solvent, surfactant, and organosiloxane polymer to achieve a desirable homogenous solution.

Table 1 provides twenty eight example silicone premix formulations containing organosiloxane polymer, surfactant, and solvent. The percent transmittance is indicative of whether the silicone premix is a homogenous silicone premix. A percent transmittance greater than 5% indicates a more preferred silicone premix. Homogenous silicone premix exhibit higher emulsion performance as illustrated by the emulsion droplet size distribution as indicated in the “Emulsion Screener” columns.

TABLE 1

| | SLM21- | Arquad | Tergitol | Ethoquad | | | | | |
|---|---|---|---|---|---|---|---|---|---|
| Example | 214 §¹ | HTL8-MS | Blend §² | C25 | Ethanol | Water | % T §³ | % <1000 nm | median |
| 1 | 80% | 12% | | | 8% | | 0.29 | 70.75 | 0.244 |
| 2 | 80% | 12% | | | | 8% | 66.43 | 68.65 | 0.174 |
| 3 | 80% | 18% | | | | 2% | 0.09 | 41.19 | 1.758 |
| 4 | 86% | 12% | | | | 2% | 0.19 | 11.07 | 9.969 |
| 5 | 88% | 6% | | | 6% | | 6.04 | 42.64 | 1.644 |
| 6 | 82% | 6% | | | 12% | | 6.44 | 33.26 | 1.815 |
| 7 | 94% | 6% | | | | | 0.06 | 26.33 | 8.431 |
| 8 | 92% | 6% | | | | 2% | 6.98 | 17.41 | 9.268 |
| 9 | 90% | 6% | | | | 4% | 8.30 | 11.89 | 12.526 |
| 10 | 97% | 3% | | | | | 4.83 | 12.89 | 13.479 |
| 11 | 95% | 3% | | | | 2% | 54.65 | 8.83 | 18.303 |
| 12 | 81% | | 15% | | | 4% | 0.11 | 74.4 | 0.272 |
| 13 | 80% | | 18% | | 2% | | 0.08 | 24.96 | 6.831 |
| 14 | 88% | | 6% | | | 6% | 95.52 | 29.49 | 9.054 |
| 15 | 90% | | 6% | | | 4% | 8.92 | 26.47 | 10.984 |
| 16 | 82% | | 6% | | 12% | | 0.63 | 9.88 | 17.503 |
| 17 | 94% | | 6% | | | | 0.06 | 9.38 | 27.105 |
| 18 | 92% | | 6% | | | 2% | 0.11 | 7.18 | 26.503 |
| 19 | 96% | | 3% | | | 1% | 0.42 | 9.93 | 21.43 |
| 20 | 97% | | 3% | | | | 2.77 | 6.73 | 55.513 |
| 21 | 81% | | | 15% | | 4% | 0.11 | 2.39 | 145.451 |
| 22 | 80% | | | 18% | 2% | | 0.09 | Fail | Fail |
| 23 | 78% | | | 6% | 12% | 4% | 67.58 | 6.69 | 41.901 |
| 24 | 88% | | | 6% | | 6% | 0.56 | 5.81 | 62.124 |
| 25 | 94% | | | 6% | | | 0.05 | 3.65 | 118.086 |
| 26 | 90% | | | 6% | | 4% | 0.07 | 2.14 | 186.36 |
| 27 | 92% | | | 6% | | 2% | 0.07 | 1.03 | 211.289 |
| 28 | 82% | | | 6% | 12% | | 0.15 | 0.97 | 170.32 |

¹SLM21-214 is an organosiloxane polymer is obtained as a 100% active and described in an example below.
²TERGITOL Blend is 65 parts TERGITOL 15S-12 and 35 parts TERGITOL 15S-5.
³greater than 5% percent transmittance indicates a homogenous silicone premix. % Transmittance is measured on a Hewlett Packard 8453 spectrophotometer at 570 nm wavelength in 4 mL plastic UV/VIS transparent cuvettes.

Examples 5, 14, and 23 of Table 1 are among the most preferred homogenous silicone premixes. Emulsification performance is evaluated qualitatively by creating a one-hundred gram sample of 20% organosiloxane polymer emulsion. The emulsion is made adding water to the homogenous silicone and premix mixing for 30 second at 3000 rpm on a FlakTech SpeedMixer™ DAC 150 FVZ-K, then observing the resultant particle size distribution. The speedmixer mixes at ˜10⁴ W/kg energy density. The temperature is ambient ˜21° C. Premix samples are placed in 100 g FlakTech SpeedMixer sample cups, and then water is added to make a target 100 g of 20% SLM emulsion. The cup is then immediately placed in the SpeedMixer and mixed at 3000 rpm for 30 seconds.

Generally, the preferred emulsification systems create a unimodal distribution, with a median particle size less than about 1000 nm. In this method, total energy applied is kept low to allow discrimination between treatments; almost all resultant particle size distributions are bimodal or trimodal. Most preferred systems have the highest % of droplets <1000 nm and the smallest overall median droplet size.

These results highlight that some solvents work better than others. In the case of Arquad® HTL8-MS, ethanol is preferred (even though water offers the ability to reach a very high % Transmittance). Arquad® HTL8-MS emulsifies more poorly in the presence of water than with no solvent, indicating water is least desirable for this system because it has higher affinity for the organosiloxane than the surfactant. Conversely, the Tergitol™ blend works best with water as solvent; ethanol has little impact. Ethoquad® C25 performs best with a mixed water/ethanol solvent system.

In one embodiment, the surfactant is TERGITOL™ and the solvent is water in a 3:1 weight ratio, respectively, resulting in a homogenous silicone premix having 92% organosiloxane polymer by weight of the homogenous silicone premix.

In another embodiment, the surfactant is ARQUAD® and the solvent is water in a 3:2 weight ratio, respectively, resulting in a homogenous silicone premix having 90% organosiloxane polymer by weight of the homogenous silicone premix.

In another embodiment, the surfactant is ETHOQUAD® and the solvent is ethanol and water such that the ratio of surfactant:ethanol:water is 3:6:2 weight ratio, respectively, resulting a homogenous silicone premix having 78% organosiloxane polymer by weight of the homogenous silicone premix.

In another embodiment, the solvent consists essentially of water and is 1%-5%, alternatively from 2% to 4% water by weight homogenous silicone premix, and about 4% to 8%, alternatively from 5% to 7%, surfactant by weight homogenous silicone premix resulting in an homogenous silicone premix having 85% to 95%, alternatively from 86% to 94%, alternatively from 88% to 93%, alternatively combinations thereof, of organosiloxane polymer by weight of the homogenous silicone premix.

In yet another embodiment, the homogenous silicone premix comprises from 85% to 94% of organosiloxane polymer, 1% to 6% water as solvent, and 4% to 9% surfactant, by weight of the homogenous silicone premix.

In yet another embodiment, the 75% to 95%, alternatively from 76% to 92% of organosiloxane polymer, 1% to 16% solvent comprising water, and 3% to 9% surfactant, by weight of the homogenous silicone premix.

### Emulsion Making

Another aspect of the invention provides for making an organosiloxane polymer emulsion by adding the homogenous silicone premix to water, preferably at ambient temperature conditions and/or at low energy density mixing conditions. The resulting organosiloxane polymer emulsion made be added and mixed with a consumer product precursor composition. In one embodiment, the organosiloxane polymer emulsion comprises from 50% to 90%, or 66% to 90%, or 70% to 85%, or 75% to 85%, or 45% to 65%, or 50% to 60%, or 50% to 55%, or combinations thereof, of organosiloxane polymer by weight of the organosiloxane polymer emulsion; and about 10% to 50% water by weight organosiloxane polymer emulsion.

Alternatively, the homogenous silicone premix may be added directly to a consumer product precursor composition, wherein the consumer product precursor composition comprises at least water and consumer product active, to obtain the organosiloxane polymer emulsion. Without wishing to be bound by theory, the emulsion performance of the homogenous silicone premix is such that the water content of the consumer product precursor is typically enough to drive emulsification. Preferably this step is conducted at ambient temperature conditions and/or at low energy density mixing conditions. In one embodiment, the consumer product comprising the organosiloxane polymer emulsion comprises from 0.1% to 20%, or 1% to 15%, or 2% to 10%, or 3% to 5%, or 0.5% to 4%, or 1% to 5%, or 2% to 6%, or combinations thereof, of organosiloxane polymer by weight of the consumer product.

The organosiloxane polymer emulsion is preferably in oil-in-water droplets such that the mean droplet diameter (as measured by volume distribution base) is below 1 micron, preferably about 0.5 microns. In one embodiment, preferably at least 80%, preferably at least 90%, preferably at least 95%, preferably at least 97%, preferably at least 98%, preferably at least 99%, preferably at least 100% droplets by volume count of the emulsion droplets of organosiloxane polymer is below 2 microns diameter, preferable below 1.5 microns, preferably below 1.0 microns, preferably below 1 micron. Particle size distribution is measured using a static laser diffraction instrument, operated in accordance with the manufactures instructions. Examples of suitable particle sizing instruments include: Horiba Laser Scattering Particle Size and Distributer Analyzer LA-930 and Malvern Mastersizer.

Without wishing to be bound by theory, the relatively small particle size may enhance the softness performance and deposition of the organosiloxane polymer to the target substrate (e.g., fabric, hair, etc.) when the consumer product is used for its intended purpose.

### Consumer Products

Another aspect of the invention provide for making a consumer product comprising the step of adding the organosiloxane polymer emulsion or homogenous silicone premix to a consumer product precursor composition. The consumer product precursor composition comprises water and at least one consumer product active. These actives are characterized by the type of consumer product. Examples may include perfume, hair shampoo actives, hair conditioning actives, cosmetic actives, beauty care actives, and surfactants (anionic, cationic, amphoteric, nonionic surfactants, and combinations thereof). The actives may comprise from 1% to 50% by weight of the final consumer product composition. Perfumes are described, e.g. U.S. Pat. No. 5,137,646. In one embodiment, the perfume includes aldehydes and ketones, including unsaturated ketones and enones such as damascene. One example is delta-damascone. The active, in one embodiment, is a fabric softening active. One class of fabric softening actives includes cationic surfactants. Examples of suitable cationic surfactants include quaternary ammonium compounds. Exemplary quaternary ammonium compounds include alkylated quaternary ammonium compounds, ring or cyclic quaternary ammonium compounds, aromatic quaternary ammonium compounds, diquaternary ammonium compounds, alkoxylated quaternary ammonium compounds, amidoamine quaternary ammonium compounds, ester quaternary ammonium compounds, and mixtures thereof. A final fabric softener composition (suitable for retail sale) may comprise from about 1% to about 30%, alternatively from about 10% to about 25%, alternatively from about 15 to about 20%, alternatively from about 1% to about 5%, alternatively combinations thereof, of fabric softening active by weight of the final fabric softener composition. Fabric softener compositions, and components thereof, are generally described in US 2004/0204337. In one embodiment, the fabric softening composition is a so called rinse added composition. In such embodiment, the composition is substantially free of detersive surfactants, alternatively substantially free of detersive anionic surfactants.

In another embodiment, the pH of the fabric softening composition is acidic, for example between pH 2 and 5.

### Organosiloxane Polymers

The organosiloxane polymers for use in the disclosed fabric care compositions may comprise

### A.

A first repeat unit of structure of Formula I:

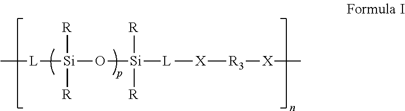

wherein:
   (i) each X may be independently selected from the group consisting of

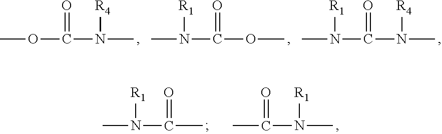

and combinations thereof;
(ii) each L may be a linking bivalent alkylene radical, or independently selected from the group consisting of

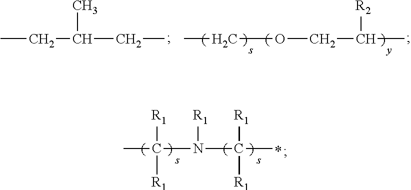

—(CH₂)ₛ—, and combinations thereof;
(iii) each R may be independently selected from selected from the group consisting of H, C₁-C₂₀ alkyl, C₁-C₂₀ substituted alkyl, C₆-C₂₀ aryl, C₆-C₂₀ substituted aryl, alkylaryl, —OR₂, and combinations thereof;
(iv) each R₁ may be independently selected from the group consisting of H, C₁-C₈ alkyl, substituted alkyl, and combinations thereof;
(v) each R₂ may be independently selected from the group consisting of H, C₁-C₄ alkyl, substituted alkyl, aryl, substituted aryl, and combinations thereof;
(vi) each R₃ may be a bivalent radical independently selected from aromatic radicals, aliphatic radicals, cycloaliphatic radicals, and combinations thereof, therein the bivalent radical may comprise from about 2 to about 30 carbon atoms; and
(vii) each R₄ may be independently selected from the group consisting of H, C₁-C₂₀ alkyl with molecular weight from 150 to 250 daltons, aryl, substituted alkyl, cycloalkyl, and combinations thereof;
(viii) p may be an integer of from about 2 to about 1000, or from about 10 to about 500;
(ix) s may be an integer of from about 2 to about 8;
(x) y is an integer of from about 0 to about 50, or about 1 to about 10;
(xi) n may be an integer of from about 1 to about 50;
B a surfactant selected from the group consisting of anionic, cationic, amphoteric, nonionic surfactants, and combinations thereof; and
C a material containing an aldehyde and/or ketone group.

In a further aspect, the organosiloxane polymer may comprise a second repeat unit of the structure of Formula II:

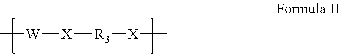

to produce a copolymer of the repeat units of the structure of Formula III

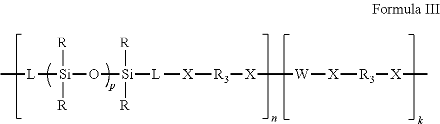

wherein:
(i) W is an alkylene radical derived from an organic molecule containing at least two functional groups selected from the group consisting of amino, hydroxyl, carboxyl, and combinations thereof;
(ii) k is an integer of from 0 to about 100.

In one aspect, R may be selected from the group consisting of methyl, ethyl, propyl, isopropyl, butyl, pentyl, hexyl, octyl, decyl, dodecyl, cycloalkyl, aryl especially phenyl, naphthyl, arylalkyl especially benzyl, phenylethyl, and combinations thereof.

In a further aspect, the fabric care composition may comprise an organosiloxane polymer having the structure of Formula III wherein:
(i.) R may be methyl;
(ii.) R₁ may be H;
(iii.) each R₂ may be independently selected from the group consisting of H, C₁-C₄ alkyl, substituted alkyl, aryl, substituted aryl, and combinations thereof;
(iv.) R₃ may be selected from the group consisting of C₂-C₁₂ C₆ alkylene radicals and combinations thereof
(v.) R₄ may be selected from the group consisting of alkyl, substituted alkyl with 1-6 tertiary amine groups with molecular weight from 140 to 250 Dalton, and combinations thereof;
(vi.) L may be

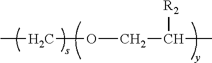

or —(CH₂)ₛ—,
(vii.) X may be selected from the group consisting of,

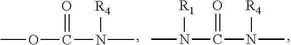

and combinations thereof;
(viii.) p may be an integer of from about 30 to about 300
(ix.) y may be an integer of from about 0 to about 50, or about 1 to about 10 and
(x.) s may be an integer of about 1 to about 50.

The second repeat unit may be added as a diluent, to modify the physical properties or alter the solubility of the organosiloxane polymer, or to improve the physical stability of the organosiloxane polymer emulsion.

In one aspect, the synthesis of organosiloxane polymer involves a conventional polycondensation reaction between a polysiloxane containing hydroxy functional groups or amine functional groups at the ends of its chain (for example, α,ω-dihydroxyalkylpolydimethylsiloxane or α,ω-diaminoalkylpolydimethylsiloxane or a aminoω-hydroxyalkylpolydimethylsiloxane) and a diisocyanate to produce the organosiloxane polymers as shown below:

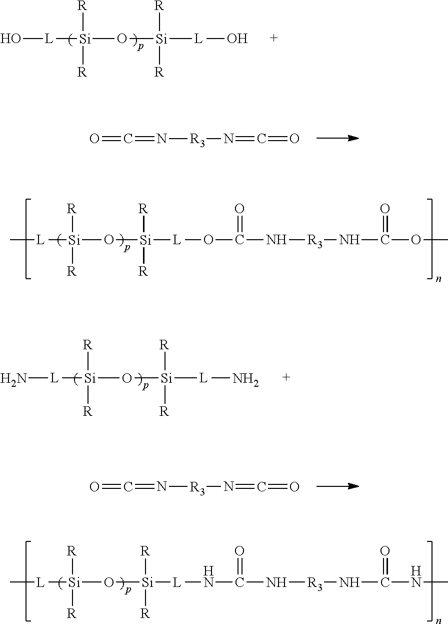

Optionally, organopolysiloxane oligomers containing a hydroxyalkyl functional group or an aminoalkyl functional group at the ends of its chain may be mixed with an organic diol or diamine coupling agent in a compatible solvent. The mixture may be then reacted with a diisocyanate. Diisocyanates that may be used include alkylene diisocyanate, isophorone diisocyanate, toluene diisocyanate, diphenylmethane diisocyanate, naphthalene diisocyanate, dicyclohexylmethane diisocyanate, xylene diisocyanate, cycloxyl diisocyanate, tolylene+diisocyanate, and combinations thereof. In one aspect, the alkylene diisocyanates include hexamethylene diisocyanate, butylene diisocyanate, or mixtures thereof.

In one aspect, the organosiloxane polymers of Formula III have a random distribution of first and second repeat units. In another aspect, polysiloxane may be used in stoichiometric excess such that the organosilicone polymer produced may comprise a polysiloxane at each end. In a second aspect, isocyanate may be used in stoichiometric excess such that the organosiloxane polymer produced has an isocyanate group at each end of the polymer chain, producing a diisocyanate. In such case, the organosiloxane polymer is reacted in a second step with a coupling agent to produce a polysiloxane polymer of Formula III. The polysiloxane polymer made using the two-step process generally has longer blocks of polysiloxanes joined together by one or more coupling agents.

Suitable coupling agents include organic molecules that contain at least two groups capable of reacting with an isocyanate group under appropriate reaction conditions. In one aspect, the coupling agents are selected from the group consisting of diols, polyols, polyetheramines, aminoalcohols, diamines, polyamines, chain extenders, crosslinkers, dispersion stabilizers, chain blockers, and combinations thereof, such as those described in Szycher's Handbook of Polyurethanes by Michael Szycher, CRC Press (1999). Suitable diols include di, tri and polyhydric alcohols, for example ethylene glycol, 1,3-propanediol, 1,4-butanediol, 1,5-pentanediol, 1,6-hexanediol, neopentyl glycol, 3-methyl-1,5-pentanediol, 1,7-heptanediol, 1,8-octanediol, 1,9-nonanediol, 1,10-decanediol and 1,12-dodecanediol, cyclohexanedimethanol, alkyl propane diol and their derivatives, and combinations thereof. Suitable polyols include polyether polyols, polyester polyols, and polycarbonate polyols. Polyether polyols include glycols with two or more hydroxy groups, such as those made by ring-opening polymerization and/or copolymerization of ethylene oxide, propylene oxide, trimethylene oxide, tetrahydrofuran and 3-methyltetrahydrofuran. In one aspect, polyether polyols include polyalkylene glycol, polyethylene glycol, polypropylene glycol, polybutylene glycol and their copolymers, polymers of tetrahydrofuran and alkylene oxide, Poly BD and polytetramethylene etherglycol (PTMEG) and combinations thereof. Suitable polyester polyols include polyalkylene terephthalate, polyalkylene isophthalates polyalkylene adipate, polyalkylene glutarate, or polycaprolactone. Suitable polycarbonate polyols include those carbonate glycols with two or more hydroxy groups, produced by condensation polymerization of phosgene, chloroformic acid ester, dialkyl carbonate or diallyl carbonate and aliphatic polyols. Suitable polyols for preparing the polycarbonate polyols include diethylene glycol, 1,3-propanediol, 1,4-butanediol, 1,5-pentanediol, 1,6-hexanediol, neopentyl glycol, 3-methyl-1,5-pentanediol, 1,7-heptanediol, 1,8-octanediol, 1,9-nonanediol, 1,10-decanediol and 1,12-dodecanediol. Polyetheramines are based on polyetherpolyols in which the terminal hydroxyl group is replaced by amine groups. The polyetheramine backbone, in one aspect, may be based on polyalkylene oxide, for example, propylene oxide, ethylene oxide, or mixtures thereof. Other backbone segments may be included, or the reactivity of the polyetheramine may be varied by hindering the primary amine or through secondary amine functionality. Suitable polyetheramines include those commercially available from Huntsman Chemicals of Woodlands Tex. under the trade name Jeffamine® Suitable diamines, polyamines, or aminoalcohols include linear or branched or cyclic diamines, triamines, aminoalcohols, alkylene diamines, dialkylenetriamine and mixtures thereof. In one aspect, the diamine may be selected from the group consisting of 2-methylpentamethylenediamine, bishexamethylenetriamine, diaminocyclohexane, ethylenediamine, propylenedimine pentanediamine, hexamethylenediamine, isophoronediamine, piperazine, and combinations thereof. These may be sold under the trade name Dytek® (by Invista of Wilmington, Del.). Aminoalcohols include diamines with 2-12 carbon atoms which also have one or more hydroxyl groups in their structure.

Additional coupling agents, which may be useful in increasing the stability of the polymer dispersion in an aqueous environment, include difunctional reactants with hydroxyl or amine groups and one or more anionic, cationic, or amine group selected from the group consisting of —COO⁻, —SO₃⁻, —OSO₃⁻, —OPO₃⁻, —N(R₅)₂ or —N⁺(R₅)₃X⁻, and combinations thereof, wherein each R₅ is selected from the group consisting of hydrogen; C₁-C₂₀ alkyl, benzyl or their substituted derivatives, and combinations thereof, and wherein X⁻ is any compatible anion.

The organosiloxane polymer may also contain a monofunctional chain-blocker (also referred to as a “capping group”). Monofunctional chain blockers, as used herein, are coupling agents containing a single group capable of reacting with an isocyanate group. The monofunctional chain blocker can be used to regulate the molecular weight of the polymer. Suitable chain blockers may include C₂-C₄ dialkylenetriamine and its derivatives, bis(2-dialkylaminoalkyl)ether; N,N dialkylethanolamine, Pentaalkyldiethylenetriamine; Pentaalkyldipropylenetriamine; N,N-dialkylcyclohexylamine, N,N,N′-trialkyl N′ hydroxyalkylbisaminoethyl ether; N,N-bis(dialkylaminopropyl)-N-isopropylamine; and N,N,N′-trialkylaminoalkylethanolamine. In one aspect the polyamine may be selected from the group consisting of N,N-bis(3-dimethylaminopropyl)-N-isopropanolamine, bis(2 dimethylaminoethyl)ether, N,N-dimethylethanolamine, pentamethyl diethylenetriamine, N,N,N′,N′,N′-pentamethyldipropylenetriamine, N,N,N′-trimethyl-N′-hydroxyethyl bisaminoethylether, N,N-bis(3-dimethylaminopropyl), N-isopropanolamine, N-(3-dimethylaminopropyl)-N,N-diisopropylamine, 1,3 propanediamine, N′(3-(dimethylamino)propyl)-N,N-dimethyl, N,N,N′-trimethylaminoethyl ethanolamine, and combinations thereof.

In one aspect, the organosiloxane polymer may be terminated with a monofunctional chain blocker to produce a structure:

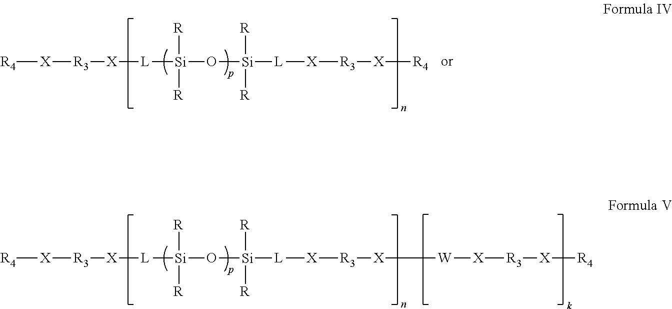

wherein, R₄ may be selected from the group consisting of C₁-C₂₀ alkyl, substituted alkyl group, and combinations thereof, wherein at least about 50% of the R₄ groups have one or more tertiary amino groups. R, R₃, X, L, n, W, and k are defined as above.

In one aspect, the weight average molecular weight of organosiloxane polymer may be from about 1000 to about 500,000 Daltons, or from about 2,000 Daltons to about 250,000 Daltons.

### EXAMPLES

Example 1 is SLM 21-214—an example of an organosiloxane. Two equivalents of α,ω-dihydrogenpolydimethylsiloxane (Available from Wacker Silicones, Munich, Germany), having degree of polymerization of 50, is mixed with 4 equivalents of 2-hydroxyethyl allyl ether and heated to 100° C. A catalytically amount of Karstedt's catalyst solution is added, whereupon the temperature of the reaction mixture rises to 119° C. and a clear product is formed. Complete conversion of the silicon-bonded hydrogen is achieved after one hour at 100 to 110° C. Two equivalents of N,N-bis(3-dimethylaminopropyl)isopropanolamine (Jeffcat® ZR50 available from Wacker Silicones, Munich, Germany) and 3 equivalents of hexamethylenediisocyanate (HDI) are then meteringly added in succession at a reaction temperature of 120° C. Urethane formation is then catalyzed with a catalytic amount of di-n-butyltin dilaurate. After the batch has been held at 120° C. for 3 hours it is cooled down, forming a very viscous liquid.

### Example 2

### Making a Homogenous Silicone Premix

400 g of SLM21-214® polymer is stirred under overhead mixing at rpm resulting in vortexing. Separately, 4.17 g water, 4.38 g Tergitol™ 15S-5, and 8.13 g Tergitol™ 15S-12 are mixed by hand. This surfactant/solvent premix is added to the SLM21-214®, adjusting the mixing for the viscosity change and to maintain vortex mixing. After 3 minutes mixing, the mixing is turned off and the solution is allowed to stand 1 hour. A homogenous silicone premix is obtained.

### Example 3

### Making a Organosiloxane Polymer Emulsion

A homogenous silicone premix from Example 2 is added to water 21 parts to 79 parts, respectively. An IKA® ULTRA TURRAX T25 is used at 9500 Hz (setting #2) using S 25 N or S 25 G dispersing element for 2 minutes. The coarse emulsion is added to Panda 2K valve homogenizer at 700 bar in single valve mode, 5 passes to obtain a organosiloxane polymer.

### Example 4

The organosiloxane emulsion of example 3 is added a fabric softener active composition comprising a bis-(2-hydroxyethyl)-dimethylammonium chloride fatty acid ester having an average chain length of the fatty acid moieties of from 16 to 18 carbon atoms, to make a final fabric softener composition comprising 2% of the organosiloxane emulsion and 15% of the ester by weight of the fabric softener composition. Perfume and other adjuncts are added.

### Example 5

Phase Inversion in Batch, benchtop scale (110 g batch). A jar-6 cm diameter, 7.8 cm high (150 ml volume) is obtained. An IKA® RW20 w/small paddle (5 cm dia) is also obtained. A 20:2.5 (or 88.9% active) solution of SLM 21-214® in ethanol by blending with Ika® overhead mixer for ˜15 min. 22.5 g of the silicone/ethanol and add 1.75 g active Arquad® HTL8-MS (2.1 g of 85% active) and stir with Ika® mixer at ˜270 rpm for 15 min. Add water (room temp) in 4 approximately equal (˜18 g) portions, stifling at least 15 minutes between additions. Total amount of water added=74 g. After the final addition is stirred ˜15 minutes, adjust pH with glacial acetic acid. 1.4 g is added to bring the pH to 4.5. Particle size (Horiba) analysis indicates Mean and Median PS of ˜300 nm.

### Example 6

Direct Emulsification, benchtop scale (500 g batch). The formulation is: 20.0% SLM21-214 (active); 1.05% Tergitol 15S-5 (surfactant); 1.95% Tergitol 15S-12 (surfactant); 2.50% Ethanol (diluent); 0.60% Glacial Acetic Acid (pH adjust to ˜3.5). Firstly, SLM21-214®, acid and Ethanol in beaker, stir until combined. The surfactants and water are prepared as a premix. Heat may be used to help dissolve. Let cool. Add surfactant mix and water mix to SLM21-214®. Use Ika® T25 High Shear Homogenizer at setting 2 (9500 Hz) for 3 minutes to emulsify. Mix acid in slowly to adjust pH to 3.5.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of making a consumer product comprising the steps:
(a) providing a surfactant/solvent mixture;
(b) providing an organosiloxane polymer;
(c) mixing the surfactant/solvent mixture and the organosiloxane polymer to obtain a homogenous silicone premix;
(d) adding the homogenous silicone premix to a consumer product precursor composition to obtain the consumer product.

2. The method of claim 1, further comprising mixing the premix and the consumer product precursor composition with an energy density lower than 10⁵ W/kg, and wherein the consumer product precursor composition comprising a consumer product active and water.

3. The method of claim 2, wherein the step of adding the homogenous silicone premix to the consumer product precursor composition is conducted under ambient temperature conditions.

4. The method of claim 3, wherein the homogenous silicone premix comprises comprising from 85% to 94% of the organosiloxane polymer, 1% to 6% water as solvent, and 4% to 9% surfactant, by weight of the homogenous silicone premix, a percent transmission of the percent transmittance of light is greater than 50 using a 1 centimeter cuvette at a wavelength of 570 nanometers.

5. The method of claim 3, wherein the homogenous silicone premix emulsifies in the consumer product to form oil-in water emulsion droplets such that the mean droplet diameter (as measured by volume distribution base) is below 1 micron.

6. The method of claim 5, wherein the solvent comprises water, and wherein the weight ratio of surfactant to water in the surfactant/solvent mixture is from 5:1 to 1:2, respectively.

7. The method of claim 6 wherein the mixing the surfactant/solvent mixture and the organosiloxane polymer to obtain a homogenous silicone premix is conducted under ambient temperature.

8. The method of claim 6, wherein mixing the surfactant/solvent mixture and the organosiloxane polymer obtains a homogenous silicone premix, and is conducted under low energy density mixing conditions.

9. The method of claim 8, wherein at least 80 droplets by volume count of the oil-in-water emulsion droplets of organosiloxane polymer are below 1 microns diameter.

10. The method of claim 9, wherein the organosiloxane polymer comprising at least one repeat unit having the structure of Formula (I): 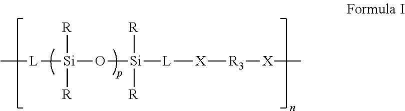
wherein:
(i) each X is independently selected from the group consisting of 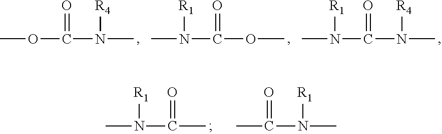
and combinations thereof;
(ii) each L is a linking bivalent alkylene radical, or independently selected from the group consisting of 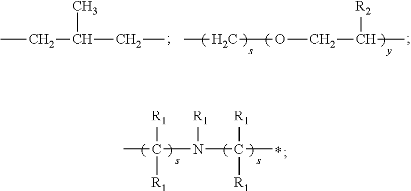
—(CH₂)ₛ—; and combinations thereof;
(iii) each R is independently selected from the group consisting of H, C₁-C₂₀ alkyl, C₁-C₂₀ substituted alkyl, C₆-C₂₀ aryl, C₆-C₂₀ substituted aryl, alkylaryl, —OR₂ and combinations thereof;
(iv) each R₁ is independently selected from the group consisting of H, C₁-C₈ alkyl or substituted alkyl, and combinations thereof;
(v) each R₂ is independently selected from the group consisting of H, C₁-C₄ alkyl, substituted alkyl, aryl, substituted aryl, and combinations thereof;
(vi) each R₃ is a bivalent radical independently selected from the group consisting of aromatic, aliphatic and cycloaliphatic radicals with 2 to 30 carbon atoms, and combinations thereof; and
(vii) each R₄ is independently selected from the group consisting of H, C₁-C₂₀ alkyl with molecular weight from 150 to 250 Dalton, aryl, substituted alkyl, cycloalkyl groups, and combinations thereof;
(viii) p is an integer of from 2 to 1000;
(ix) s is an integer of from 2 to 8;
(x) y is an integer of from 0 to 50;
(xi) n is an integer of from 1 to 50.

11. The method of claim 10, wherein the organosiloxane polymer comprises a second repeat unit having the structure of Formula II 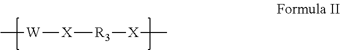
to produce a copolymer comprising the first and second repeat unit having the structure of Formula III 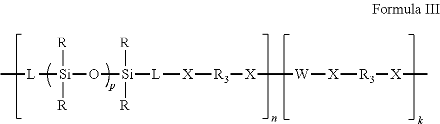
wherein:
(i) each X is independently selected from the group consisting of 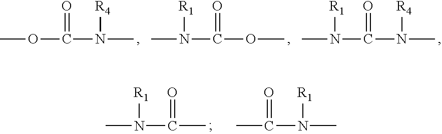
and combinations thereof;
(ii) each L is a linking bivalent alkylene radical, or independently selected from the group consisting of 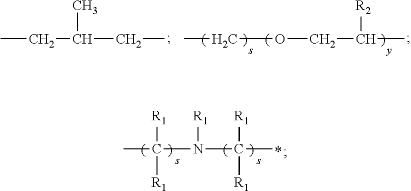
—(CH₂)ₛ—; and combinations thereof;
(iii) each R is independently selected from the group consisting of H, C₁-C₂₀ alkyl, C₁-C₂₀ substituted alkyl, C₆-C₂₀ aryl, C₆-C₂₀ substituted aryl, alkylaryl, —OR₂ and combinations thereof;
(iv) each R₁ is independently selected from the group consisting of H, C₁-C₈ alkyl or substituted alkyl, and combinations thereof;
(v) each R₂ is independently selected from the group consisting of H, C₁-C₄ alkyl, substituted alkyl, aryl, substituted aryl, and combinations thereof;
(vi) each R₃ is a bivalent radical independently selected from the group consisting of aromatic, aliphatic and cycloaliphatic radicals with 2 to 30 carbon atoms, and combinations thereof; and
(vii) each R₄ is independently selected from the group consisting of H, C₁-C₂₀ alkyl, aryl, substituted alkyl, cycloalkyl groups, and combinations thereof;
(viii) s is an integer of from 2 to 8;
(ix) y is an integer of from 0 to 50;
(x) n is an integer of from 1 to 50
(xi) k is an integer selected from 0 to 100; and
(xii) W is an alkylene radical derived from an organic molecule containing at least two groups selected from the group consisting of amino groups, hydroxyl groups, carboxy groups and mixtures thereof.

12. The method of claim 11, wherein consumer product active is 1% to 49% of a surfactant selected from the group consisting of anionic, cationic, amphoteric, nonionic surfactants, and combinations thereof, by weight of the consumer product composition; and the consumer product comprises from 0.1% to 10% of the organosiloxane polymer.

13. The method of claim 10, wherein the consumer product active is a fabric softener active comprising from 3% to 25% of an ester quaternary ammonium compound by weight of the fabric softener product composition.

14. A method of making a consumer product comprising the steps:
(a) providing a homogenous silicone premix comprising from 75% to 95% of a organosiloxane polymer, 1% to 16% solvent comprising water, and 3% to 9% surfactant, by weight of the homogenous silicone premix, wherein the homogenous silicone premix comprises a percent transmission of the percent transmittance of light is greater than about 50 using a 1 centimeter cuvette at a wavelength of 570 nanometers;
(b) adding to the homogenous silicone premix to a consumer product precursor composition under ambient temperature conditions;
(c) mixing the premix and the precursor under low energy density conditions to form the consumer product, wherein the consumer product comprises oil-in-water organosiloxane polymer emulsion droplets such that the mean droplet diameter (as measured by volume distribution base) is below 1 micron;
wherein the organosiloxane polymer comprising at least one repeat unit having the structure of Formula (I): 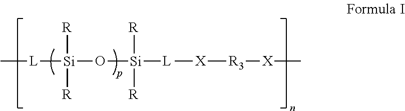
wherein:
(i) each X is independently selected from the group consisting of 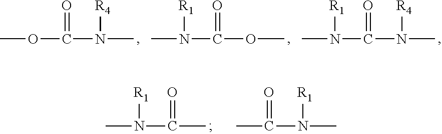
and combinations thereof;
(ii) each L is a linking bivalent alkylene radical, or independently selected from the group consisting of 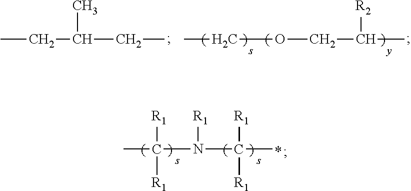
—(CH₂)ₛ—; and combinations thereof;
(iii) each R is independently selected from the group consisting of H, C₁-C₂₀ alkyl, C₁-C₂₀ substituted alkyl, C₆-C₂₀ aryl, C₆-C₂₀ substituted aryl, alkylaryl, —OR₂ and combinations thereof;
(iv) each R₁ is independently selected from the group consisting of H, C₁-C₈ alkyl or substituted alkyl, and combinations thereof;
(v) each R₂ is independently selected from the group consisting of H, C₁-C₄ alkyl, substituted alkyl, aryl, substituted aryl, and combinations thereof;
(vi) each R₃ is a bivalent radical independently selected from the group consisting of aromatic, aliphatic and cycloaliphatic radicals with 2 to 30 carbon atoms, and combinations thereof; and
(vii) each R₄ is independently selected from the group consisting of H, C₁-C₂₀ alkyl with molecular weight from 150 to 250 Dalton, aryl, substituted alkyl, cycloalkyl groups, and combinations thereof;
(viii) p is an integer of from about 2 to about 1000;
(ix) s is an integer of from about 2 to about 8;
(x) y is an integer of from about 0 to about 50;
(xi) n is an integer of from about 1 to about 50.

15. The method of claim 14, wherein the consumer product composition comprises:
(a) from 0.1% to 50% of the organosiloxane polymer by weight of the consumer product composition, and wherein the organosiloxane polymer comprises a second repeat unit having the structure of Formula II 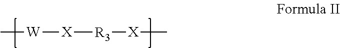
to produce a copolymer comprising the first and second repeat unit having the structure of Formula III 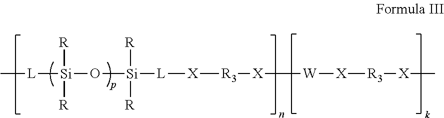
wherein:
(i) each X is independently selected from the group consisting of 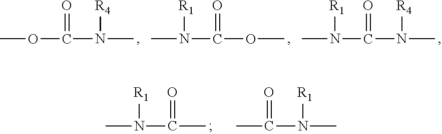
and combinations thereof;
(ii) each L is a linking bivalent alkylene radical, or independently selected from the group consisting of 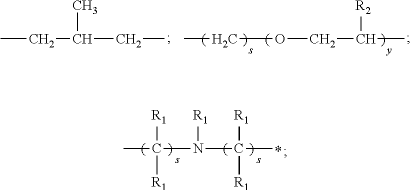
—(CH₂)ₛ—; and combinations thereof;
(iii) each R is independently selected from the group consisting of H, C₁-C₂₀ alkyl, C₁-C₂₀ substituted alkyl, C₆-C₂₀ aryl, C₆-C₂₀ substituted aryl, alkylaryl, —OR₂ and combinations thereof;
(iv) each R₁ is independently selected from the group consisting of H, C₁-C₈ alkyl or substituted alkyl, and combinations thereof;
(v) each R₂ is independently selected from the group consisting of H, C₁-C₄ alkyl, substituted alkyl, aryl, substituted aryl, and combinations thereof;
(vi) each R₃ is a bivalent radical independently selected from the group consisting of aromatic, aliphatic and cycloaliphatic radicals with 2 to 30 carbon atoms, and combinations thereof; and
(vii) each R₄ is independently selected from the group consisting of H, C₁-C₂₀ alkyl, aryl, substituted alkyl, cycloalkyl groups, and combinations thereof;
(viii) s is an integer of from 2 to 8;
(ix) y is an integer of from 0 to 50;
(x) n is an integer of from 1 to 50
(xi) k is an integer selected from 0 to about 100; and
(xii) W is an alkylene radical derived from an organic molecule containing at least two groups selected from the group consisting of amino groups, hydroxyl groups, carboxy groups and mixtures thereof;
(b) wherein consumer product active is 1% to 49% of a surfactant selected from the group consisting of anionic, cationic, amphoteric, nonionic surfactants, and combinations thereof, by weight of the consumer product composition

16. A method of making a consumer product:
(a) mixing a polyorganosiloxane with a high hydrogen bonding solvent (i.e., having a Hansen Solubility Parameter of greater than 10.0 for Hydrogen bonding capacity, δ_{H}), to obtain a binary homogenous silicone premix;
(b) adding the binary homogenous silicone premix to an aqueous surfactant mixture to obtain a polyorganosiloxane emulsion, wherein the surfactant comprises of an asymmetric di-hydrocarbyl quaternary ammonium compound of formula (II): 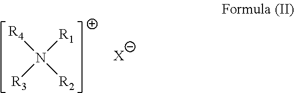
wherein R₁ comprises a C₁₂ to C₂₂ hydrocarbyl chain, wherein R₂ comprises a C₆ to C₁₂ hydrocarbyl chain, wherein R₁ has at least two more carbon atoms in the hydrocarbyl chain than R₂, wherein R₃ and R₄ are individually selected from the group consisting of C₁-C₄ hydrocarbyl, C₁-C₄ hydroxy hydrocarbyl, benzyl, —(C₂H₄O)ₓH where x has a value from 1 to 10, and mixtures thereof, and wherein X⁻ is a anion;
or a polyalkoxy quaternary ammonium compound of Formula (III) 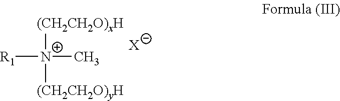
wherein x and y are each independently selected from 1 to 20, and wherein R1 is C6 to C22 alkyl;
(c) optionally mixing the aqueous surfactant mixture while the homogenous silicone premix is added to the aqueous surfactant mixture.

17. The method of claim 16, wherein the emulsion comprises from 50% to 90% by weight of the emulsion of water.

18. The method of claim 17, wherein the emulsion comprises from 10% to 50% by weight of the emulsion of the polyorganosiloxane.

19. The method of claim 18, wherein the weight ratio of the surfactant to polyorganosiloxane from 1:1 to 1:30, respectively.

20. The method of claim 19, wherein the consumer product composition comprises:
(a) from 0.1% to 50% of the organosiloxane polymer by weight of the consumer product composition, and wherein the organosiloxane polymer comprises a second repeat unit having the structure of Formula II 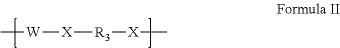
to produce a copolymer comprising the first and second repeat unit having the structure of Formula III 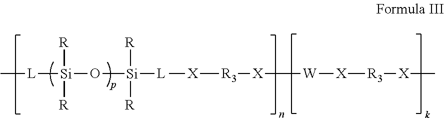
wherein:
(i) each X is independently selected from the group consisting of 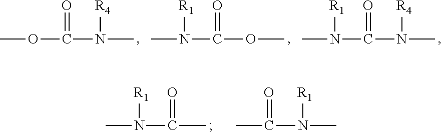
and combinations thereof;
(ii) each L is a linking bivalent alkylene radical, or independently selected from the group consisting of 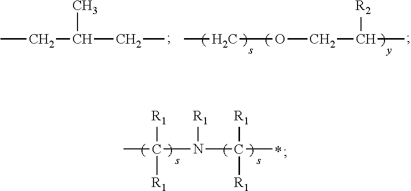
—(CH₂)ₛ—; and combinations thereof;
(iii) each R is independently selected from the group consisting of H, C₁-C₂₀ alkyl, C₁-C₂₀ substituted alkyl, C₆-C₂₀ aryl, C₆-C₂₀ substituted aryl, alkylaryl, —OR₂ and combinations thereof;
(iv) each R₁ is independently selected from the group consisting of H, C₁-C₈ alkyl or substituted alkyl, and combinations thereof;
(v) each R₂ is independently selected from the group consisting of H, C₁-C₄ alkyl, substituted alkyl, aryl, substituted aryl, and combinations thereof;
(vi) each R₃ is a bivalent radical independently selected from the group consisting of aromatic, aliphatic and cycloaliphatic radicals with 2 to 30 carbon atoms, and combinations thereof; and
(vii) each R₄ is independently selected from the group consisting of H, C₁-C₂₀ alkyl, aryl, substituted alkyl, cycloalkyl groups, and combinations thereof;
(viii) s is an integer of from 2 to 8;
(ix) y is an integer of from 0 to 50;
(x) n is an integer of from 1 to 50
(xi) k is an integer selected from 0 to about 100; and
(xii) W is an alkylene radical derived from an organic molecule containing at least two groups selected from the group consisting of amino groups, hydroxyl groups, carboxy groups and mixtures thereof;
(b) wherein consumer product active is 1% to 49% of a surfactant selected from the group consisting of anionic, cationic, amphoteric, nonionic surfactants, and combinations thereof, by weight of the consumer product composition

21. A method of making a consumer product comprising the steps:
(a) mixing a polyorganosiloxane with a high hydrogen bonding solvent (i.e., having a high Hansen Solubility Parameter specifically, having a Hansen Solubility Parameter of greater than 10.0 for Hydrogen bonding capacity, δ_{H}) to obtain a binary homogenous silicone premix;
(b) slowly adding a surfactant to the binary homogenous silicone premix to obtain a homogenous silicone premix, wherein the surfactant comprises of an asymmetric di-hydrocarbyl quaternary ammonium compound of formula (II): 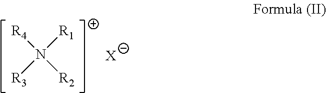
wherein R₁ comprises a C₁₂ to C₂₂ hydrocarbyl chain, wherein R₂ comprises a C₆ to C₁₂ hydrocarbyl chain, wherein R₁ has at least two more carbon atoms in the hydrocarbyl chain than R₂, wherein R₃ and R₄ are individually selected from the group consisting of C₁-C₄ hydrocarbyl, C₁-C₄ hydroxy hydrocarbyl, benzyl, —(C₂H₄O)ₓH where x has a value from 1 to 10, and mixtures thereof, and wherein X⁻ is a anion; or 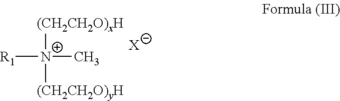
wherein x and y are each independently selected from 1 to 20, and wherein R1 is C6 to C22 alkyl preferably C12 to C18 alkyl, branched or unbranched, and X⁻ is a anion;
wherein the aqueous surfactant mixture comprises a surfactant/polyorganosiloxane weight ratio of from 1:1 to 1:30, alternatively from 1:1 to 1:10;
(c) adding water to the homogenous silicone premix to obtain a polyorganosiloxane emulsion;
(d) optionally mixing the homogenous silicone premix while the water is added to obtain the emulsion.

22. The method of claim 21, wherein the emulsion comprises from about 50% to 90% by weight of the emulsion of water.

23. The method of claim 21, wherein the emulsion comprises from 10% to 50% by weight of the emulsion of the polyorganosiloxane.

24. The method of claim 21, wherein the weight ratio of the surfactant to polyorganosiloxane from 1:1 to 1:30, respectively.

25. The method of claim 16, wherein at least 80 droplets by volume count of the oil-in-water emulsion droplets of organosiloxane polymer are below 2 microns diameter.

26. The method of claim 20, wherein at least 80 droplets by volume count of the oil-in-water emulsion droplets of organosiloxane polymer are below 2 microns diameter.
